(19) United States
(12) Patent Application Publication
(10) Pub. No.: US 2010/0112558 A1
(43) Pub. Date: May 6, 2010

(54) Probe Bead Synthesis and Use

(75) Inventors: Xiaolian Gao, Houston, TX (US); Xiaochuan Zhou, Houston, TX (US)

Correspondence Address: Xiaolian Gao, 2212B Bellefontaine, Houston, TX, 77030, US

(73) Assignee: XIAOLIAN GAO, HOUSTON, TX (US)

(21) Appl. No.: 12/264,242

(22) Filed: Nov. 3, 2008

(51) Int. Cl. C12Q 1/68; G01N 33/566

(52) U.S. Cl. 435/6; 435/7.5

(57) ABSTRACT

The present invention relates to the field of methods and devices of miniaturized synthesis. More specifically, the present invention relates to the parallel synthesis of large number of different types of molecules and oligomers, such as oligonucleotides (oligos), peptides, lipids, carbohydrates, small ligand molecules, and other organic and inorganic molecules as probes for multiplexing assays. The probes may be synthesized from and/or attached to nanobeads to microbeads. The present invention provides for assays of multiplexing large scale biology, such as analysis of genomic DNAs and RNAs and proteomic proteins or peptides performed simultaneously on the synthetic beads.

### FIELD OF THE INVENTION

The present invention relates to the field of methods and devices of miniaturized synthesis. More specifically, the present invention relates to the parallel synthesis of large number of different types of molecules and oligomers such as oligonucleotides (oligos), peptides, lipids, carbohydrates, small ligand molecules, and other organic and inorganic molecules as probes for multiplexing assays. The probes may be synthesized from and/or attached to nanobeads to microbeads. The present invention providesassays of multiplexing large scale biology, such as analysis of genomic DNAs and RNAs and proteomic proteins or peptides performed simultaneously on the synthetic nanobeads.

### BACKGROUND OF THE INVENTION

Research in large scale biology is moving towards nano-scale and single molecule experiments. Decoding genomic information and the messages of cells and living organisms requires millions to billions of measurements from a single object, such as a cell, an animal or an individual. Even with the progress made in recent years, present day assays would consume impossibly large reagent volumes reagents, require immense storage facilities, and need an army of robotic instruments to perform the large scale experiments required by modern genomic and proteomics. Miniaturization of experimental devices, samples, and assays is important to maximize the number of experiments that can be performed simultaneously.

Genomics, proteomics and other fields of large-scale biology involve parallel studies of a large number of biomolecules. Large scale biological assays such as genomic DNA assays may require hundreds of thousands to millions of analyses of individual DNA sequences. Proteomic protein assays may likewise requirelarge numbers of analyses. Assays also need to be carried out in tens of thousands tests; therefore, the total number of analyses is also very large, in hundreds of thousands to millions. In practice, it is therefore necessary that these studies are carried out in a miniaturized and multiplexing (multiple analysis/reactions in parallel) format to allow high throughput and low reagent consumption. One such example is seen in the field of nucleic acid analysis. Rapid progress in genomics DNA sequencing technologies (Margulies et al 2005, Nature 437, 376-380; Mardis, E. R. 2008, Human Genetics 9, 387-402; herein incorporated by reference) (e.g. 454 Life Sciences' GS FLX system, Illumina/Solexa's GA system and ABI's SOLiD technology), genomic assays, multiplexing DNA synthesis, and bioinformatics technologies has enabled researchers to obtain in-depth molecular pictures of complex biological systems of human and other life forms.

In the last decade, hybridization using DNA microarrays has been the dominant method for large scale analysis of genes and DNA mutations, Lockhart et al. 1995, Nat. Biotech. 14, 1675-1680; Schena et al. 1995, Science 270, 467-460; herein incorporated by refrenece), but sequencing of nucleic acids has many advantages over hybridization for analysis of DNA and RNA. Direct sequencing does not require prior sequence information for setting up hybridization assays, nor does sequencing require labeling with detection tags (e.g. fluorescence, quantum dots, luminescence) as hybridization based analysis does. Sequencing also provides a direct reading of the sequence rather than an indirect answer which is obtained by reading the sequence of the hybridization probes. Sequencing thus enables the generation of more accurate information about genomic content. Finally, sequencing also measures the number of sequences analyzed, and the results thus obtained are digital rather than those converted from analog image signals to digital. Sequencing results are more quantitative than hybridization. Prior art methods of sequencing have limited throughput (e.g. one sample per run and 96-runs in parallel using a capillary sequencer). These early generation sequencing methods can, therefore, not be applied to genome-scale DNA and RNA analysis which requires multiplexing, rapid, high-throughput methods.

Multiplexing sequencing of a biological sample may be random(Margulies et al. 2005, Nature 437, 376-380; herein incorporated by reference). This means there is no selection for the sample content nor specific sequence selection. Rather selection is based onconventional size-selection, charge or polarity selection, or other selection criteria based on chemical or physiochemical properties of the analyte molecules (i.e., DNA and RNA). Electrophoresis, chromatography, filtration, and other separation methods well known to those skilled in the art have been used. However, the sample complexity and thus the number of analyte DNA and RNA sequences for multiplexing sequencing may be reduced by selecting one or more subgroups of the sequences.

The selection can be achieved by sequence-specific hybridization using probe sequences. Probe sequences or probes (DNA or RNA or a chimera of DNA and RNA sequences) are normally complementary to the target or analyte sequences (these are the sequences to be sequenced) in a sample. Through hybridization, the hybridized sequences in a sample can be selectively isolated and sequenced in subsequent steps (sequence-specific enrichment); or the hybridizing sequences in a sample may be excluded from the subsequent sequencing steps (sequence-specific depletion). The probes for sequence-specific selection are determined by the sequencing needs and are designed accordingly using the base complementary rules of nucleic acid hybridization. For instance, Tn oligonucleotides (n=number of residues, ranging from 8 to 200 preferably 15-50) are probes for selection for An-containing target sequences, and oligos which are hybridized specifically to p53 genes in a sample are probes for these genes. The genomic locations such as a specific sequence, or exons or the junctions of exon and intron can be selected using probes hybridizing.

Probes have been immobilized on a planar surface as probe microarrays or on a spherical surface as probe beads. These forms of probes have been widely used in microarray applications (Lockhart et al. 1995, Nat. Biotech. 14, 1675-1680; Schena et al. 1995, Science 270, 467-460; herein incorporated by reference) for gene expression, comparative genomics (CGH), gene copy number variations (CNVs), chromatin immunoprecipitation (chIP) and chip-hybridization for identification and profiling of DNA binding regulation sites, DNA methylation analysis, single nucleotide polymorphism (SNP) analysis.

Probe beads are molecular carriers which are used in or out of solutionand in single or multiple forms for detection, isolation solid supports, affinity binding media, and/or detection tags. Probe beads have been used in a wide variety of applications, such as bead microarrays using pre-synthesized oligos Gunderson, K. L. et al. 2005, Nat Genet 37, 549-554; herein incorporated by reference) using pre-synthesized oligos. These syntheses were accomplished on micro-well trier plates, where reagents were added separately to each reaction well in which one oligo was synthesized. Genomic sequencing technology employs millions of beads as random sequence loaders and is used by the 454 sequencing method (www.454.com). These applications demonstrated a transition from nanometer to micron-scale experiments which requires, probe bead dimensions in the nanometer to the low micron scale. These technological advancements demonstrate promise in not only low cost human genome sequencing, but also for general bioassays.

There are however, still areas for improvement in current large-scale biological assays. Present sequencing methods sequence target sequences by probability or on a random basis. There is no guarantee that a specific sequence will be captured and therefore multiple (usually 10-20×) sequencing runs are required to ensure reasonably complete coverage of target sequences. This level of redundancy and the considerable associate with it makes it highly desirable that each reaction effectively focus on the genomic regions of interest. Efficient sequencing is also important because DNA is full of repeats and sequences of unknown functionality. Many sequencing experiments have specific areas of interest, such as genes related to cancer, which is a small fraction of the entire genome. In addition, the region of interest varies widely with each area of research. For example, sequencing regions of coding or non-coding sequences (e.g. small RNA, intronic, intergenic, untranslated), SNP, regulatory regions (replication, transcription and/or translation regulatory, other genetic function regulatory), areas of imprinting/methylation, transpliced and transposon regions, or any combination of these can be of interest. DNAs of different organelles may also be of interest. Clearly, the lack of effective and low cost sampling methods limits the use of the genomic sequencing technology for many routine target-specific re-sequencing applications Overcoming the limitations of the present methods of sequencing would be a tremendous step forward in fully realizing the potential of sequencing technologies for general research and clinical laboratorial applications. One immediate impact would be in improving the specificity, sensitivity and reliability of DNA analysis in SNP and epigenetic assays. The improvements would permit gene expression profiling using sequencing reactions rather than DNA microarrays. Given the advantages of sequencing over microarray hybridization, the replacement of the hybridization-based microarrays by sequencing represents a significant technology advancement.

There are other advantages of a bead-based process in biological sample experiments. One such example is the use of magnetic beads (Leach, L. et al. 1994, Placenta 15, 355-364; Georgieva, J. et al. 2002, Melanoma Res 12, 309-317; Zhorowski, M. et al. 2005, Methods Mol Biol 295, 291-300; Thaxton, C. S. et al. 2006, Clin Chim Acta 363, 120-6; Nakanishi, H., et al. 2007, Oncol Rep 17, 1315-1320; herein incorporated by reference). In application examples, the magnetic). Magnetic beads of a few microns in diameter may be derivatized with binding molecules such as streptavidin protein. Such magnetic beads may be capture materials (while streptavidin is a capture molecule which binds to ligand molecule in solution and thus allowing separation of the binding molecules from solution) for capturing molecules labeled with biotin in a sample. The magnetic beads can be retained by a magnetic source while the solution containing these beads can be removed and exchanged. In this fashion, the molecules bound to the capture magnetic beads can be easily separated from the molecules in solution. Compared to electrophoresis; which involves cutting the correct gel band and eluting the desired molecules, the use of magnetic beads is much faster and simpler. Magnetic beads can be derivatized with different capture molecules based onseparation requirements. The use of capture molecules and probe molecules is equivalent.

Nanobead synthesis methods and device will significantly enhance our ability and efficiency to collect biological information at genome and transcriptome scales much like ultra-high speed processing and mega storage capacity to computer and electronic industry that have led to today's wide spread use of personal electronics devices. This will eventually help us to understand systems biology at an unprecedented level and further the connection of biological sciences with personal health. The methods of the present invention will provide means to significantly enrich the content of molecular probes and lower the barriers for comprehensive biological assays and personalized genomics and medicine.

A few examples for areas of impact should help to explain the point. First, it has become clear that unless the current genomic tests can be run simpler, more economically and at speeds at least an order of magnitude faster, human genome-based comprehensive studies will be stalled due to prohibitively large amount of reagents and solutions will be required. Many of these large volume screening assays, such as population-based single nucleotide polymorphism (SNP) biomarker analysis for cancer, are currently carried out only at large core facilities of few universities or medical centers and are available to only a limited of number of people. There is a need to make the assays available to average research laboratories so that large populations can be tested. This will, for instance, greatly increase the chance that millions of the human genome SNPs will be fully analyzed. Human genome is estimated to have 4 million SNPs. Even with 1% of world population of 6.7 billion tested we would need to perform 10¹⁵ tests, assuming the use of 3 to 4 redundancy passes. Gene copy number variations (CNVs), gene reallocations and fusions, and other forms of genomic aberrant changes are also areas where high-throughput sequencing is required. The present invention makes population genome analysis practical. The probe bead-based assays will consume much smaller volumes of sample and reagents and offer solutions to the existing barrier.

Current biomolecular array tools are made from a single type of probe molecule per hybridization site/spot, i.e. DNA oligomers (DNA oligos) in situ synthesized or pre-synthesized and then immobilized on surfaces (Gao et al. (2004), Biopolymers 73, 579-596; herein incorporated by reference). These narrowly defined probe contents restrict the ability to obtain information about DNA, RNA, and proteins simultaneously in a single assay, i.e., a comprehensive understanding of biological systems in which different kinds of molecules are actively interacting. The present invention provides methods and tools that enable comprehensive assays by simultaneously obtaining through probe molecules of different biomolecular classes. That is, the nanobead arrays will provide for mixed molecule arrays containing DNA, RNA, carbohydrate, peptide combinations. These probe molecules have wide applications as hybridization counterparts, aptamers, specific binding ligands, allosteric binders, etc.

High density arrays of the present invention made from carbohydrate oligomers or oligosacchrides have long been wanted for studies of a wide range of biomolecular interactions involving carbohydrate moieties ranging from cell receptors, glycosylated proteins, antibiotics, and polysaccharides. Carbohydrate arrays and arrays consisting of a combination of carbohydrates and oligomers from nucleic acids and/or peptides, such as those described in Gao, X. et al., WO2008/003100, will enable innovative experiments. The various modified peptides, such as glycol-decorated peptides prepared through click chemistry (Gao, X. et al., WO2008/003100) are suitable probe molecules.

The availability of affordable probe bead mixes will enable various target-specific genomic assays, which in turn will greatly increase the throughput of genomic experiments. The synthesis flexibility of the probe bead mixes of the present invention will allow the development of applications beyond whole genome sequencing. The present invention provides methods for sequencing specific regions in whole genome DNA or transcriptome RNA samples to obtain high quality quantitative measurements in a single reaction. This enables routine performance of large-scale studies of human genetics (SNP, CGH, Chip-on-Chip, methylation, etc.) and gene expression (coding mRNA or noncoding RNA) related to population, sex, age, disease, environmental exposure, etc. The probe bead-based assays of the present invention can be performed at significantly reduced costs, and on a much larger scale than prior art methods.

Presently, nanometer sub-micron and microbeads are available and surface modification methods have been well developed. Applications of probe beads are widespread, however, the making/synthesizing of thousands to millions of the various nano to micron probe beads in situ and in parallel and that can be addressable and containing defined content quickly, has, prior to the present invention, not been possible.

Preparing such a bead library is a new challenge. The well-known split-and-pool method produces a bead pool, but this method is not suitable to stepwise synthesis of biopolymers, such as oligonucleotides, peptides, or carbohydrates. Robotic spotting or other methods of immobilizing molecules onto the beads have been used but these methods are only practical for a bead pool of thousands of different compounds. This is because the compounds are pre-synthesized and it is cost- and time-prohibitive to post-synthesize a larger number of compounds than a few thousands. Several methods of parallel synthesis, such as light directed synthesis of oligonucleotides by Affymetrix, Nimblegen, and Febit, photogenerated reagent method by Atactic Technologies, direct reagent delivery to the reaction sites, i.e., ink-jet printing synthesis by Agilent, and eletrochemical methods by Combimatrix (reviewed by (a) Gao, X., Gulari, E., and Zhou, X., 2004, Biopolymers 73, 579-596; (b) Gao, X. et al., 2004, Molecular Diversity 8, 177-187; Fodor, S. et al., 1991, Science 251, 767-773; Hughes, T. R. et al. (2001) Nat. Biotechnol. 19, 342-347; Gao, X. et al. U.S. Pat. No. 7,211,654; Gao, X., Zhou, X., and Gulari, E. U.S. Pat. No. 6,426,184; Gao, X, Pellois, J. P., and Yao, W. U.S. Pat. No. 6,965,040 and U.S. Pat. No. 7,235,670; Gulari, E. et al. US publication 20070224616; herein incorporated by reference), are able to generate this large number of compounds. However, these syntheses are conducted on discrete flat surfaces in stead of beads; no probes can be generated from these processes. Diffraction gratings have been used to encode beads as substrates for chemical synthesis (Moon, J. et al., U.S. Pat. No. 7,190,522; herein incorporated by reference). However, in this method, the substance as substrate for synthesis is not immobilized on surface of synthesis as described in the present invention or the encoding method uses a “substantially single material”. Furthermore, the make of the gratings requires the use of specialized optical materials and raises concerns on manufacturing cost, especially when applied to genome-scale applications.

### SUMMARY OF THE INVENTION

The present invention relates to methods and miniaturized array synthesis devices, and simple, inexpensive, high throughput, and novel technology for fabrication of probe bead mixtures, i.e., thousands to millions of different nano to micron probe beads containing predetermined molecular contents. More specifically, the present invention relates to miniaturized synthesis systems for ultra fast and large scale generation of probes and probe beads which are functionalized beads of nanometer to micron sizes (nano and microbeads) and derivatized with large amounts of different types of oligonucleotides (oligos), peptides, lipids, carbohydrates, small ligand molecules, and other organic and inorganic molecules.

The present invention provides methods and devices for creation of a variety of probe beads. The probes include DNA and RNA oligonucleotides, modified DNA and RNA oligonucleotides, aptamers (folded nucleic acid oligos, structured peptides, aptamers specifically recognize certain target molecules), carbohydrates, peptides, epitopes, lipids, and synthetic molecules commonly used in the various bioassays.

In one embodiment of the present invention, a miniaturized synthesis device is used to generate oligos, peptides, and carbohydrates on nanobeads. The probe nanobeads may be generated by the synthesis of probes such as oligosand forming a bond link between the probes and the beads. Several in situ parallel synthesis methods are available for making probe (Fodor, S. et al. 1992, Science 251, 767-773; reviewed in Gao, X., Gulari, E., and Zhou, X., 2004, Biopolymers 73, 579-596, and Gao, X. et al. 2004, Nucleic Acids Res. 29, 4744-4750; herein incorporated by reference) and these) These syntheses can be performed on planar surfaces to produce probeson planar surfaces which may or may not be cleaved from the surface. The present invention provides methods for producing probe beads of nanometer to micron sizes using parallel in situ synthesis.

Probe synthesis may be accomplished by using methods for large scale parallel synthesis of microarrays (reviewed in Gao, X., Gulari, E., and Zhou, X., 2004, Biopolymer 73, 579-596; and Gao, X. et al. 2004, Mol. Dev. 8, 177-187; herein incorporated by reference). One method (Gao, X. et al. 1998, 2001, 2004) utilizesphotogenerated reagent such as an acid (PGA) to direct the parallel synthesis using conventional chemistry for oligo synthesis on microfluidic chip. An example of the synthesis chip is a pico-liter microfluidic array synthesis device depicted in FIG. 1. An example of a synthesis device of the configuration as shown in FIG. 1 is made from silicon layer (101), on which reaction sites (107 and 108; 107 sites are light irradiated), flow channels (109) contain 200 pL 128×31 μm three-dimensional reaction sites or 3D chambers (107), each of which holds solution. There are inlet and outlet ports which permit the ingress and egress of liquid (102, 106). This closed system is produced by annealing of a silicon-layer with glass layer at high temperature. In another embodiment of the present invention, oligo microarrays such as those by ink-jet method, PGA chemistry, and light deprotection of photolabile protection group for synthesis of oligos (FIG. 2) can also be used (Hughes, T. R. et al. 2001, Nat. Biotechnol. 19, 342-347; Gulari, E. et al., US publication 20070224616; Fodor, S. et al. 1992, Science, 251, 767-773; herein incorporated by reference) synthesizing oligos on glass plate surfaces (FIG. 2) can also be used).

The present invention relates to producing probe beads which may be further modified to generate a secondary generation probe beads. For instance, oligos on probe beads may be hybridized to the complementary oligos which are conjugated to protein, antibody, peptide, carbohydrate, lipid, or small molecules (Kozlov, I. A. et al. 2004, Biopolymers 73, 621-630; herein incorporated by reference). The formation of hybrid duplexes results in probe beads loaded with the conjugated molecules, forming arrays of protein, antibody, peptide, carbohydrate, lipid, or small molecules. The helices of oligos may be further stabilized by cross-linking of the two hybridizing strands so that the secondary array molecules do not dissociateunder assay conditions. The identity of the conjugate molecules can be determined by the conjugated oligos through several methods. One method is to hybridize the probe beads to an addressable bead array. A second method includes hybridization to a known oligo. Finally the conjugated oligo's identity may be ascertained by sequencing. Other probe identification methods used commonly are also suitable for obtaining information from the secondary generation probe beads.

The present invention relates to the field of combinatorial synthesis using miniaturized parallel in situ synthesis to create molecular contents on nanobeads in a miniaturized format. The synthetic molecules are conjugated to beads in four different ways: (a) by directly synthesizing probes on beads which are immobilized on surface and removing the beads from surface after the synthesis is completed; (b) by adding functionalized beads to an array of synthetic molecules to form conjugation bonds between the beads and the synthetic molecules to form one-bead-one-type-molecule probe beads (FIG. 6); (c) by mixing functionalized beads and the synthetic molecules which are detached from the synthesis surface to form a mixture of probes on beads; and (d) by directly synthesizing probes on beads using coupling-divide cycles of synthesis with a bead sorting device (FIG. 3 and FIG. 4). A common feature of the probe beads produced by the four methods is the large numbers of different probe beads suitable for large scale biology applications. A large scale biology experiment is one that simultaneously analyzes a large number of target molecules as results of: (a) a comprehensive test or (b) a largenumber of test samples. These applications require large amounts of nanobeads of various sequence content as assay probes.

The present invention also provides for inexpensive, flexible, and efficient methods for producing molecules, such as oligos. In one embodiment of the present invention, oligos are produced at a scale of at least 10-fold greater than that of microarray synthesis, in which a single area of about 100 square micron (μm²), 1 fmol or more of molecules are generated (FIG. 6). The increased amount of synthesis is obtained by increasing the reaction surface area by immobilizingbeads on the microarray synthesis surface (FIG. 7). Oligos thus synthesized are removed after synthesis and collected as a mixture for off-chip applications, such as for natural or artificial DNA synthesis, siRNA vector library construction, primers for allele specific PCR, target-specific capture of DNA sequences, in vitro and in vivo chromatin staining, probes for DNA and RNA staining, molecular cloning, molecular barcoding library, DNA assembly elements, DNA computing elements, peptide DNA library, preparation of RNA transcripts, and many other applications known to those skilled in the field. The bead-surface of a microarray synthesis device is also suitable for producing peptides, carbohydrates, and other synthetic molecules that can be produced by solid phase synthesis. In another embodiment of the present invention, molecules such as oligos are obtained by direct synthesis on encoded beads (i.e., the molecular contents of beads are trackable through reading of their signals which may be fluorescence, luminescence, electronic, magnetic, other forms of these or a combination of these forms of signals) using coupling-divide cycles of synthesis with a binary sorting bead synthesis device (FIG. 3 and FIG. 4).

The present invention also relates to the conjugation reactions for bond or binding formation between surface and oligo, oligo and tagging group, surface to bead, and bead to oligo, A number of chemical methods for conjugation are suitable choices for these purposes (Kozloy, I. A. et al., 2004, Biopolymers 73, 621-630; Soellner, M. B. et al., 2003, J. Am. Chem. Soc, 125, 11790-11791; Houseman, B. T. et al., 2002, Nat. Biotech. 20, 270-274; Farooqui, F. and Reddy, P. M., 2003, US 2003/0092901; Wang, Q. et al., 2003, J. Am. Chem. Soc, 125, 3192-3193; Clarke, W. et al., 2000, J. Chrom. A, 888, 13-22; Raddatz, S. et al., 2002, Nucleic Acids Res. 30, 4793-4802; Konecsni, T, and Kilar, F., 2004, J. Chrom. A, 1051, 135-139; herein, all references incorporated by reference).

The present invention also relates to methods of probe removal for the effective production of probe bead mixes from the array synthesis reactor. The new generation of synthesizers employed in this invention, for applications using nano-scale materials, requires efficient recovery of the materials synthesized. These compositions and methods relate to surface removal of probe bead oligos in an effective form so that the oligos can be used in subsequent applications.

The choice of probe bead compositions is based on the post-synthesis applications. An assay may include only one type of molecules and/or probe beads such as those derivatized with oligos of different sequences. An assay may also include selections of different types of probe beads made from molecules of various categories such as those derivatized with DNA/RNA oligos or peptides to create novel multi-molecular content multi-purpose assay tools for analyzing and identifying analyte target molecules of various types. Assays utilizing multi-molecular contents can be accomplished in fewer steps than required by the conventional individual assays. The multi-molecular content multi-purpose assay tools uniquely allow simultaneously analysis of nucleic acids, proteins and other types of biomolecules by using a mix of probe beads of different molecular entities.

The probe beads provided by the present invention can be manufactured to serve diverse assay requirements. At least two types of probe bead synthesis can be utilized in the present invention. Probe bead devices based on microarray synthesis can be used to generate nanometer to micron probe beads without barcoded beads. A bead-sorter system generates nanometer to millimeter size probe beads. These beads can be barcoded and traceable (i.e., the content of the probe, such as sequence of the oligonucleotide or the peptide can be identified by the barcode). Probes are useful for either on-bead or off-bead applications.

In one application where a sufficient large amount (e.g. nmols) of probes is needed; probes are generated using the bead-sorting synthesis system (FIG. 3 and FIG. 4). The system has a multi-level binary tree fast sorting fluid structure and uses barcoded detectable signal to make selections. Preferably, the various forms of paramagnetic beads are synthesis substrates; alternatively, electrical, optical, thermal, morphology, molecular and combination of these physical and chemical property measurements are sensors which can be used for reading bead barcode. Electro-magnetic field generators can be used for steering bead flow directions. Barcode is a unique identification indicator relating to a molecular moiety. However, barcodes can also be used in combination to form patterns such as one barcode represents a 1-state and a second different type of barcode represents 0-state. The detection of more than one barcodes for a bead is also a means of bead identification.

Probe molecules is a form of synthesis products and thus are synthesized and then cleaved from beads for use. In another embodiment of the present invention, probe beads are more suitable choice and beads are also isolation (e.g. magnetic beads) and/or detection (e.g. fluorescence) tags. Probes attached to beads can also function as dentrimers which have the benefit to provide a multivalency effect for binding and for detection.

The present invention provides for assays of large scale biology such as genomic DNAs and RNAs and proteomic proteins or peptides performed simultaneously on the synthetic probe nanobeads. These nanobeads are created according to design considerations at unprecedented fast speed arid low cost, allowing routine large scale analysis and identification of target molecules in biological and chemical samples by direct contact or indirect contact between the samples and the probes on beads.

The application of the present invention relates to a probe bead mix for target-specific DNA and RNA analysis of specific disease genes and disease pathway-related genes, such as cancer genes, immunoresponsive genes, cardiovascular system related genes, cell development and growth regulation genes, drug metabolism-related genes (P450 genes), and many other pathway and activity connected genes, which are known to those skilled in the art.

The application of the present invention relates to a probe bead mix for emulsion PCR on a target-specific basis (Margulies et al. 2005, Nature 437, 376-380; herein incorporated by reference). DNA replication primers designed specifically for a target sequence or randomly distributed over a sequence region (FIG. 8, 801) are present on probe beads (FIG. 8, 802). Multiplexing amplification may follow the allele specific amplification using common primers (FIG. 8, 803). Replication of the analyte DNAs in many cavities/drops formed under emulsion conditions (e.g. micelles and vessels in a mixture of mineral oil and water) effectively allow target-specific DNA and RNA analysis of a set of selected disease or disease-related genes, such as cancer genes, immunoresponsive genes, cardiovascular system related genes, cell development and growth regulation genes, drug metabolism-related genes (P450 genes), and many other pathway and activity connected genes. The reduction of the overall numbers of the sequence analyses required allows a higher redundancy of sequencing and thus more reliable and reproducible results.

The application of the present invention relates to multiplexing assays of target-specific DNA and RNA analysis. In a high capacity experiment such as genomic sequencing, multiple selected sets of target molecules from multiple samples are mixed/pooled. Each selected set of target molecules from a sample is differentiated by a short stretch of two or more nucleotides inserted into the sequences of the selected set of target molecules. This nucleotide barcode is unique to each designated set of target molecules and readable by sequencing or hybridization. The number of different sets of the target molecules in a sample and the number of samples mixed/pooled in an assay are greater than one; a total of 2 or more target molecule sets can be analyzed in one assay reaction. These multiplexing assays increase throughput and reduce cost by many folds.

The application of the present invention also provides for multiplexing assays of target-specific DNA and RNA analysis using other methods such as hybridization, ligation, restriction enzymatic cleavage, nuclease enzymatic cleavage, and DNA methylation, other than sequencing.

The application of the present invention can be used in conjunction with preparation of droplets containing synthesized probes. The method utilizes the RainDance droplet technology (http://www.raindancetechnologies.com/applications/next-generation-sequencing-technology.asp) where an oligo-containing surfactant/water droplet of picoliter sizes forms from a microfluidic device. Coalescing of the probe droplet with one or more target molecules also in the form of droplet allows further manipulation of the sample for genomic analysis of the target molecules. The formation of the probe droplet may be made such that each droplet contains the or more pre-calculated copies of probes or that each droplet contains one or more pre-calculated probe beads. In one form of reaction, the probe droplets interact with target molecules in solution and thus separate the target molecules which interact with probes in droplet from those which do not interact with probes. These probe droplets have applications similar to probe beads and allowing efficient, large scale, parallel chemical and biochemical assays.

### DESCRIPTION OF THE DRAWING

FIG. 1 is a schematic drawing of a pico-liter microfluidic array synthesis device.

FIG. 2 is a planar glass plate for array synthesis.

FIG. 3 is a schematic drawing of a binary bead sorting synthesis system.

FIG. 4 is a schematic drawing of an exemplary bead synthesis system and process.

FIG. 5 is an illustration of a synthetic probe synthesized on surface.

FIG. 6 is a schematic illustration of one embodiment of an oligo probe bead molecule.

FIG. 7 is a microscope image of a reaction chamber filled with reaction beads.

FIG. 8 is an illustration of probe beads as amplification primers.

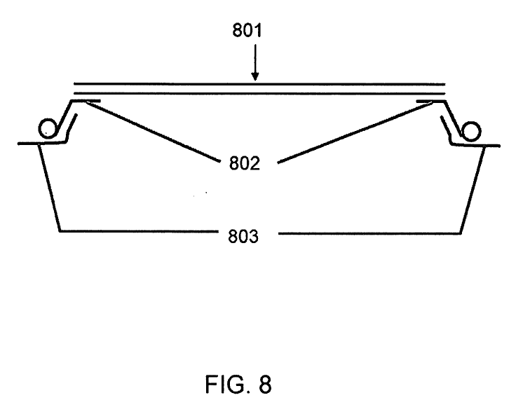

FIG. 9 is an image of beads on surface

FIG. 10 is an experimental flow comparing the results of using or without using magnetic streptavidin bead for oligo mixture processing.

### DETAILED DESCRIPTION OF THE INVENTION

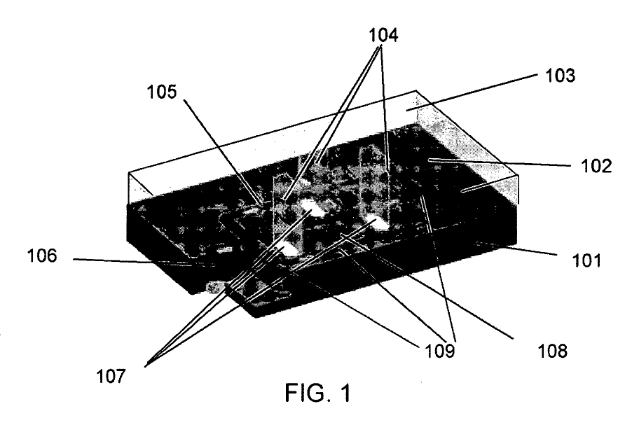

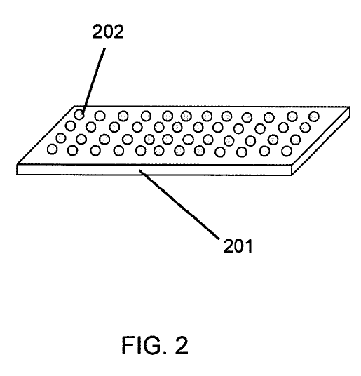

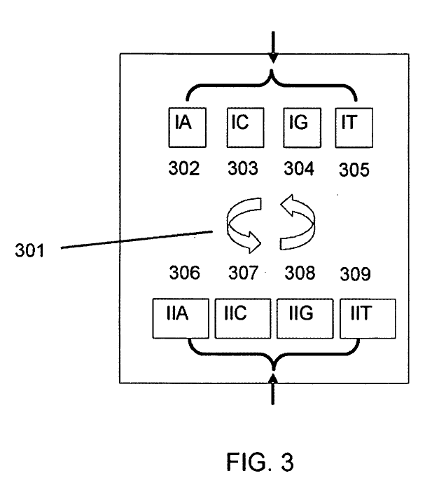

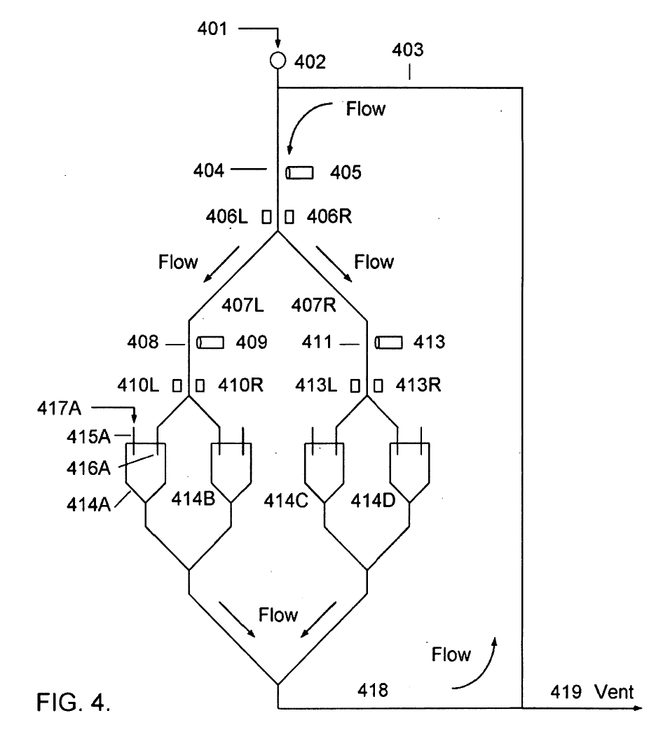

The present invention provides methods and devices for large scale, parallel making of probes and probe beads. In a preferred embodiment of this invention, the method for synthesis of probes is miniaturized in situ synthesis in an array format (FIG. 1 and FIG. 2). Thousands to tens of thousands of probes are synthesized simultaneously in fmol to pmol amounts per each probe and these probes are attached to bead materials to give probe beads. The probes can be but are not limited to DNA, RNA, carbohydrate, peptide, lipid, and small molecules and other chimera of the molecules useful for bioassays. In another embodiment of this invention, a binary sorting synthesis system (FIG. 3 and FIG. 4) and method are provided for rapid parallel synthesis of probes on beads, which are digitally barcoded such that a specific probe be synthesized on each bead according to design. This synthesis method uses beads from nanometers to millimeters in diameter and produces probes in fmol to nmol amounts for each. The present invention provides versatile products for diverse applications of genomics and the related fields of large scale biology.

In this invention, probe synthesis is carried in devices which offer surfaces that can accommodate arrays of molecules. An array contains at least 400 different probes in a square centimeter area, preferably more than 1,000 different molecules in a square centimeter area. Each type of probes is produced in sub-fmols to nanomols concentration, preferably in pmols concentration. FIG. 1 is a drawing of a microfluidic pico-liter array synthesis device (Zhou, X. et al. 2004, Nucleic Acids Res. 32, 5409-5417; herein incorporated by reference). The synthesis of probes may be carried out in parallel in the 200 pL reaction chamber for each probe. At the completion of the synthesis, probes are derivatized with a long linker group bearing a functional group to form a conjugate with the functional group of beads to form probe beads.

In a preferred embodiment of the present invention, a synthesis device such as that shown in FIG. 1. contains about 4,000 reaction chambers. A synthesis device of the type may contain a smaller (i.e., several hundreds) or larger number of reaction chambers (i.e., tens of thousands or more). These reaction chambers may contain a number of beads such as 10 μm Tantagel beads (Polymere GmbH) in reaction chambers. The surface capacity of such a bead allows for more than 10 pmol of molecules to be synthesized, which is about 10,000 fold larger than the capacity of a planar reaction cell of dimension 90×200 μm².

Another method of making probe beads entails adding the units of the sequence (such as nucleotide monomer or amino acids) one by one to the tagged bead and introducing a sorting step between each addition. The sorting step sequesters all the beads which will be subject to the same treatment in the next step, after which the beads can be re-sorted for the next step.

For example, FIG. 3 and FIG. 4 show a preferred method of oligonucleotide nanobead synthesis in which a given molecule can be addressed to a particular tagged bead. Beads can be tagged in a variety of ways including but not limited to fluorescence, radio frequency, molecular tags, molecular sequence tags, optical, magnetic, optomagnetic and combinations thereof. In this method of synthesizing oligo nanobeads, 4 reaction chambers (FIG. 3. 302 to 305) are filled with tagged, derivatized nanobeads (e.g. OH functionalized Tentagel (10 μm) beads). Each reaction chamber corresponds to one of the four DNA nucleotides A, T, G or C. After a given nucleotide is added to each bead in the reaction chamber, the beads are re-sorted into reaction chambers corresponding to the next nucleotide to be added to the growing sequence. For example, in FIG. 3 eight sequences are listed (see Sequence List). These sequences correspond to eight different molecules to be made. In the first cycle of the 3′-5′ synthesis (the methods of the present invention are not limited by the direction of the synthesis) the nanobeads corresponding to sequences #4 and #8 will start in the chamber IA (FIG. 3. 302) where an adenosine (A) monomer will be added to these beads. In like manner, beads that correspond to sequence #1 will be placed in reaction chamber IC (FIG. 3. 303), beads that correspond to sequences #2, #3, #5 and #6 will be placed in reaction chamber IT (FIG. 3. 305) and beads that correspond to sequence #7 will be placed in reaction chamber IG (FIG. 3. 304). Nucleotides corresponding to the reaction chamber will be added to the beads. In a preferred embodiment of the present invention the nucleotide monomers are conventional monomers which are 5′-DMT protected. After the coupling reaction is complete the beads are then sorted in a process that redistributes the beads in reaction chamber corresponding to the second nucleotide of the desired sequence. For example, in FIG. 3, the beads corresponding to sequence #1 are removed from reaction chamber IC (FIG. 3. 303) and distributed into reaction chamber; IIG (FIG. 3. 308) wherein a guanosine nucleotide will be added to the molecule. Beads corresponding to sequence #2 are removed from reaction chamber IT (FIG. 3. 305) and distributed into reaction chamber IIG (FIG. 3. 308) wherein a guanosine nucleotide will be added to the molecule. Beads corresponding to sequence #3 are removed from reaction chamber IT (FIG. 3. 305) and distributed into reaction chamber IIA (FIG. 3. 306) wherein an adenosine nucleotide will be added to the molecule. Beads corresponding to sequence #4 are removed from reaction chamber IA (FIG. 3. 302) and distributed into reaction chamber IIT (FIG. 3. 309) wherein a thymidine nucleotide will be added to the molecule. Beads corresponding to sequence #5 are removed from reaction chamber IT (FIG. 3. 305) and distributed into reaction chamber IIC (FIG. 3. 307) wherein a cytosine nucleotide will be added to the molecule. Beads corresponding to sequence #6 are removed from reaction chamber IT (FIG. 3. 305) and distributed into reaction chamber IIG (FIG. 3. 308) wherein a guanosine nucleotide will be added to the molecule. Beads corresponding to sequence #7 are removed from reaction chamber IG and distributed into reaction chamber IIT (FIG. 3. 309) wherein a thymidine nucleotide will be added to the molecule. Beads corresponding to sequence #8 are removed from reaction chamber IA (FIG. 3. 302) and distributed into reaction chamber IIC (FIG. 3. 307) wherein a cytosine nucleotide will be added to the molecule. The synthesis and sorting cycles are repeated until the desired sequences are synthesized.

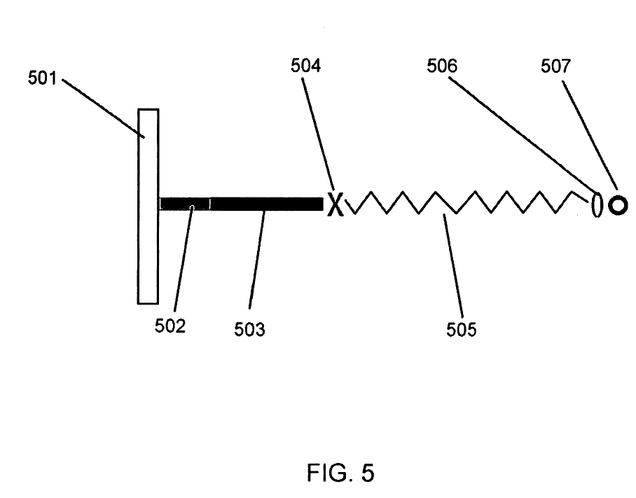

The method of the present invention is not limited by the type of molecules that have been discussed. In preferred embodiments of the present invention DNA, RNA, peptides and carbohydrates or any other molecule that is amendable to in situ synthesis may be synthesized on addressable nanobeads. The methods of synthesis of the present invention are also not limited by the number of reaction chambers that can be utilized in the synthesis of molecular nanobeads. While a single reaction chamber was utilized in the example in FIG. 5, multiple reaction chambers for each monomer species to be added can also be envisioned. Reaction chambers might also be use for more than a single step. Other synthesis protocols including the use of dimer and trimers or longer elements might also be utilized.

The number of different elements to be added will define the minimum number of reaction chambers necessary to have one reaction chamber per element. For example if the synthesis is of a peptide sequence then utilizing the naturally occurring amino acids, 20 different reaction chambers might be necessary for synthesis depending on the length of the sequence.

The synthesis device can have either isolated reaction chambers where the chambers can be physically sealed from one another or the device may have fluid connections between the reaction chambers wherein the beads can flow through a sorting device and be redistributed into other reaction chambers that are in fluid connection with the sorting device.

The addressable nanobeads of the present invention may have a density of 1-1,000,000 molecules per bead. In certain preferred embodiments the nanobead has a single molecule adhered to it.

Nanobeads and other nanoparticles can be modified so that the beads can be sorted by flow cytometry which takes advantage of the rapid (10⁷/min) bead-sorting instruments to generate pools of pre-sorted beads based on a defined set of properties of beads. Such a pool of pre-sorted beads overcomes limitations of the prior art which requires a high level of redundancy in random arrays assembled from a mixture of molecular beads. Pre-sorted beads permit specific beads to be selected for addressable nanoarrays and/or a pool of beads of known sequence contents for specific applications.

The tagged beads may be made into a variety of shapes including but no limited to cylindrical, tubular, spherical, hollowed spherical, elliptic, and disk like. The beads may contain recess structures or areas for protecting active surface moieties from physical contact with other subjects or beads. For example, the beads can be made into dumbbell shape having an active surface area in mid section while both ends of the dumbbell being coated with an inert material. The recessed structures may help avoid bead coagulation and/or damage of active surface moieties. A preferred size of the beads is from 1 nanometer to 1 centimeter in the longest dimension. A more preferred size is from 10 micron to 5 millimeter.

The tagged beads may be made from a variety of materials including but not limited to glass, ceramic, polymer, metal, semiconductor, and combination of more than one material. For example, a bead may contain a paramagnetic core encapsulated with a polymer material. The paramagnetic core facilitates transportation, sorting, and holding of the bead using magnetic force. Another exemplary bead contains a paramagnetic coating, at least on one or more sections of the bead, also to facilitate bead manipulation by magnetic force. Yet another exemplary bead contains a solid core, such as glass, that is encapsulated with a layer of polymer matrix material for increasing synthesis load. The matrix material includes but is not limited to low cross-linked polystyrene, polyethylene-glycol, and various copolymer derivatives (F. Z. Dorwald “Organic Synthesis on Solid Phase: Supports, Linkers, Reactions”, Wiley-VCH, 2002; herein incorporated by reference).

The tag marks on the beads may be produced using a variety of processes that are well-known to those who are skilled in the field of micro-fabrication. One exemplary process is laser marking. Laser marking is well known to those who are skilled in the field of laser processing (J. C. Ion “Laser Processing of Engineering Materials”, Elsevier Butterworth-Heinemann, 2005; herein incorporated by reference). An iron film is coated on a glass fiber by electroplating or by sputtering. The preferred film thickness is between 5 nm to 5 μm. The film coating is well-know to those skilled in the art of thin-film fabrication (R. L. Cornstock “Introduction to Magnetism and Magnetic Recording”, John Wiley & Sons, Inc., New York. 1999; herein incorporated by reference). Optical tags in form of coaxial ring barcodes are then laser marked on the fiber surface by ablating the iron film. The fiber is then coated with a protective thin silica film, either by vapor deposition or by sol-gel process (M. A. Aegerter “Sol-Gel Technologies for Glass Producers and Users”, Kluwer Academic Publishers, 2004; herein incorporated by reference). The fiber is cleaved or cut to form a cylindrical bead. The bead is then either derivatized with an appropriate linker moiety or coated with a matrix polymer material. The method shown above is only one exemplary illustration among many variations of bead making processes. For example, the polymer or metal fiber or wire can be used as the core of the bead. The iron film can be replaced with a paramagnetic iron oxide or nickel phosphorus film. A dark color metal oxide film can be deposited on top of magnetic film to produce a high-contrast barcode by laser marking. The fiber can be cleaved or cut after linker derivatization or matrix polymer coating. The coating of a fiber with a matrix polymer can be done in a similar way as that of putting a cladding layer on glass fiber for making optical fibers.

FIG. 4 is a schematic diagram of an exemplary binary sorting synthesis system. The system uses magnetic beads that contain optical barcodes. Before the start of a synthesis, a group of beads, each having a known barcode, is selected. Each bead is assigned with a sequence to be synthesized. At the beginning of a synthesis reaction beads-containing solution 401 is sent into the system through an entrance port 402. When a bead passes through detection port 404 its barcode is read by optical sensor 405. Depending on the barcode and its designated sequence, electro-magnetic field generator 406L or 406R is activated to cause the bead flowing either into flow channel 407L or into channel 407R so as to complete level one sorting. Level two sorting is done in a similar fashion and through detection ports 408 and 409, optical sensors 409 and 413, and electro-magnetic field generators 410L, 410R, 413L, and 413R. The bead is eventually steered into a designated reaction chamber (414A, 414B, 414C, or 414D) in which a specific sequence residue is to be added to the molecular moiety on the bead. While not shown in the FIG., a mechanism is available in each reaction chamber to hold the bead inside the chamber. Exemplary holding mechanisms include but are not limited to mechanical stoppers and magnetic fields. When all the beads have been sorted and placed into designated reaction chambers reaction reagents (e.g. 417A) are sent into the reaction chambers (414A, 414B, 414C, and 414D) through reagent deliver lines (e.g. 415A) to carry out a synthesis cycle. Reacted reagents are discharged through venting line 419. Upon the completion of the synthesis cycle beads are released from all reaction chambers and are pushed into a circulation line 418. With venting line 419 closed (venting valve is not shown in the FIG.), the beads are then returned back to level one sorting through returning line 403. The next sorting and synthesis cycle can then begin. The synthesis cycles are repeated until all designated sequences are synthesized. The present invention may be used with any known solid-phase and combinatorial synthesis process (U.S. Pat. No. 7,190,522 and references; herein incorporated by reference).

The flow channels shown in FIG. 4 can be made of glass, plastic, silicon, or any appropriate materials. The size of the channels may vary from sub-micrometer to millimeters in diameter depending on applications. For synthesis on small beads, the preferred flow channel diameter is between about 1 to about 200 micrometers. The channels can be fabricated using etching process on glass or silicon wafers. Reaction chambers (414A, 414B, 414C, and 414D) can be formed on the same wafers. For synthesis on larger beads, such as matrix polymer encapsulated beads, the preferred flow channel diameter is between about 100 micrometers to about 1 millimeter. Conventional tubing, made of glass, fluoropolymers, or other types of chemical resistant materials can be used. Reaction chambers (414A, 414B, 414C, and 414D) can be made of chemical resistant polymers such as fluoropolymers and polyphenylene sulfides, glass, or stainless steels.

The binary sorting synthesis system shown in FIG. 3 and FIG. 4 is only one exemplary illustration among many variations. For example, a buffer chamber can be placed between returning line 403 and detection port 404 to better regulate bead flow. A movable frit filter disc can be placed at the bottom of each reaction chamber (414A, 414B, 414C, or 414D) and a reagent delivery line can be placed below the filter while substrate beads lay above the filter. With this arrangement, a chamber reactor operates in a float-bed manner and good mass transfer can be achieved during synthesis reactions. Additional sorting levels can be added to meet the requirement of additional distinct residues such as in case of peptide synthesis. In a preferred mode, optical sensors 405, 409, and 413 are photodiodes. In another preferred mode optical sensors 405, 409, and 413 are CCDs (charge-coupled devices). In certain operational modes, for example when bead flow rate inside sorting channels is stable or predictable or when the time interval between two adjacent beads are sufficiently long so that the second bead enters into detection port 404 after the first bead has entered its designated reaction chamber, only one optical sensor 405 may be needed. While not shown in the figure, illumination lights may be used in conjunction with optical sensors. The optical sensors (405, 409, and 413), magnetic field generators (406L, 406R, 410L, 410R, 413L, and 413R) and fluid controls valves (hot shown in FIG. 4) can be in communication with one or more computers and their signal collection and/or movement actuations are controlled by the computer. Other bead encoding and decoding methods can be used. For example, magnetic encoding and decoding methods can be used; In this case, a magnetic recording head is placed on the side wall of a flow channel. Binary codes can be written or read to or from a paramagnetic film coated bead in the same way as that of digital recording using one or more magnetic taps or discs.

Beads can be manipulated by forces or effects other than or in addition to magnetic force. For example, using piezoelectric devices, mechanical deformation can be created inside fluid channels so as to steer the flow direction of beads. Heat, produced by laser or resistive elements, can be applied to flow channel wells and to cause flow disturbance so as to affect the flow direction of beads. A computer controlled 1D or 2D transportation arm in conjunction with a code reading device can be used to deliver tagged beads to designated reaction chambers instead of using the binary tree sorting mechanism shown in FIG. 4. The present invention significantly increases the speed of synthesis by reducing the overall operation steps and using the advanced microparticle sorting technologies. Bead selection at each reaction cycle for synthesis is processed at a speed hundreds to million per second.

In an embodiment of the present invention, after the completion of synthesis of all designated sequences, the barcoded beads can be used for performing assays on the bead surfaces or can be used for producing materials by cleaving the synthesis products from the beads. The matrix polymer encapsulated beads are particularly suitable for producing off-bead synthesis products. Individual sequence products can be produced by placing the barcoded beads into cleavage reaction wells, which can be in 96-well format, 384-well format, 1536-well format, or certain custom-made format, and perform cleavage reaction in parallel. The placement of the barcoded beads can be done using a computer controlled transportation arm in conjunction with a code reading device. A mixture product can be obtained by placing all or a selected number of beads in a cleavage reaction well and performing a cleavage reaction. These syntheses produce fmol to nmol per sequence materials, preferably, pmol to a few nmol of materials with a few thousandth or less solvent consumption as conventional one-by-one oligo synthesis such as that process used by Illumina (www.illumina.com) to produce oligo beads for bead microarrays.

In this invention, beads for loading probes have various properties. The sizes of beads preferably are in the range of a few nanometers to millimeters, and beads of one micron or so are preferably used in the array synthesis device. Beads of a few micron to millimeter diameter are preferably used in the binary sorting synthesis system. The shape of beads or nano- and mciro-particles can be spherical, elongated, cylindrical, and other irregular shapes. At the bead surface there can be coating layers of porous and/or non-porous particles to give desirable surface synthesis and/or attachment properties. The surface can be functionalized as carriers of assay probes. Different kinds of beads are applicable for making probe beads, including but not limited to silica beads (e.g. those from Bands Laboratories, Inc.), magnetic beads (e.g. those from Invitrogen/Dynal beads), polymeric beads (e.g. those from Rapp Polymers). In the present invention four types of beads and the corresponding chemistry are preferred: gold or gold coated spheres (10-100-nanometer, thiol group), avidin/streptavidin coated magnetic beads (<10 μm, biotin group), TentaGel beads (Rapp Polymere GmbH, Germany, 1-100 μm, 3, 10, 30 μm, NH2 or OH conjugation chemistry), Sephadex beads (20-50, 40-120 μm, carboxyl, NH2 conjugation chemistry). Beads may contain tags/markers for detection and identification, such as fluorescence molecules (Fluoresbrite polystyrene beads (Polysciences), luminescence molecules, chromophore molecules, magneto electronic group/print, quantum dots, biotin, etc. In this invention, beads used in the microfluidic array reactor shown in FIG. 1 are made of stable materials including, CPG (controlled pore glasses), cross-linked polystyrene, and various resins that are commonly used for solid-phase synthesis and analysis.

The present invention relates to solid surface (FIG. 5, 501) synthesis of probe molecules which may contain surface linker and spacer groups such as alkyl, polyethylene glycosyl chains. The linker group (FIG. 5, 501) is an anchor point for attachment on surface and spacer (FIG. 5, 502) provides the accessibility and structural flexibility for probes (FIG. 5, 505) to interact with target molecules. Probe molecules may contain tags (FIG. 5, 507) through conjugation (FIG. 5, 506), such as those fluorescence molecules, chromophore molecules for detection, biotin which can link to a detection molecule, or a bead moiety (FIG. 5, 507). Probes may be cleaved at a specific cleavage point (FIG. 5, 504). In one embodiment of the present inventionthe cleavage point (504) is dU (cleavable using USER kit from New England Lab (NEB)), conjugation site (506) is a biotin and streptavidin linkage and this is linked to a nanobead (507) which is linked to streptavidin.

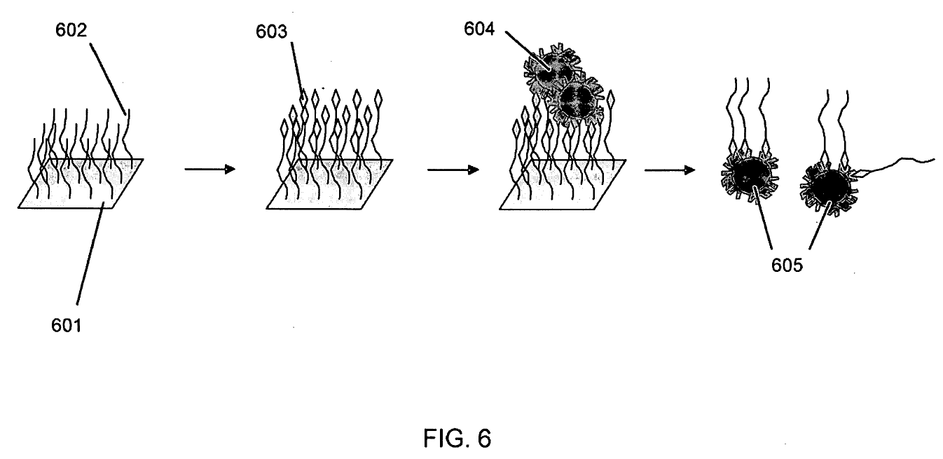

The present invention also relates to the conjugation reaction for joining two kinds of molecules, or a molecule with beads, or beads with surface. Specifically, oligos can be attached to a surface or beads and beads in solution attached to the surface oligos. Bead surface reactions are traditionally carried out using molecules in solution and functionalized to react with a bead surface. A number of chemical methods for conjugation are suitable choices for these purposes (Kozlov, I. A. et al., 2004. Biopolymers 73, 621-630; Soellner, M. B. et al., 2003, J. Am. Chem. Soc., 125, 11790-11791; Houseman, B. T. et al., 2002, Nat. Biotech. 20, 270-274; Farqoqui, F. and Reddy, P. M., 2003, US 2003/0092901; Wang, Q. et al., 2003, J. Am. Chem. Soc, 125, 3192-3193; Clarke, W. et al., 2000, J. Chrom. A, 888, 13-22; Raddatz, S. et al., 2002, Nucleic Acids Res. 30, 4793-4802; Konecsni, T, and Kilar, F., 2004, J. Chrom. A, 1051, 135-139; herein all incorporated by reference). In one embodiment of the present invention, an array of more than 100 oligonucleotides is synthesized on surface and the terminal group, preferably the 5′-terminal group, is an alkylbiotin. A solution of streptavidin coated magnetic beads (e.g. Dynabeads® M-270 Streptavidin) is added to the surface. Biotin and streptavidin are high affinity binding pairs (Kd>10¹³ M) and the solution and surface contact results in the beads binding to oligos on surface. In case when the dimension of a reaction site of oligo synthesis is much greater that the size of the bead, one bead will be surrounded by the same oligos in the reaction site (FIG. 6). In certain embodiments the biotinylated oligos that are conjugated to strepavidin beads are the same sequence to give one-bead-one-type of oligo probe beads.

The present invention also relates to the conjugation reaction for joining two molecules, or a molecule with beads, or beads with surface. Specifically, oligos can be attached to a surface or beads and beads in solution attached to the surface oligos. The conjugation reactions can occur between a pair of reactants (the first and the second functional groups from the pair of reactants) and also between multiple pairs of reactants (the third and the fourth functional groups of the second pair of reactants). The functional groups include reactive groups and high affinity binding groups, such as alkynyl, alkylazide, amino, hydroxyl, thiol, aldehyde, phosphoinothioester, maleimidyl, succinimidyl, isocynate, ester, hydrazine, strepavadin, avidin, neuavidin and biotin binding proteins. In a conjugation reaction, wherein the first functional group is biotin and the second functional group is strepavadin, avidin, neuavidin; or other biotin binding proteins; in another conjugation reaction, wherein the first functional group is alkynyl and the second functional group is azide; in another conjugation reaction, wherein the first functional group is amino and the second functional group is ester, succninimidyl, or isocynate; in another conjugation reaction, wherein the first functional group is thiol and the second functional group is phosphoinothioester, maleimidyl; in another conjugation reaction, wherein the first functional group is hydroxyl, and the second functional group is ester, succinyl, succninimidyl, or isocynate; in another conjugation reaction, wherein the first functional group is aldehyde, and the second functional group is amine, or hydrazine. For the pair of functional groups, e.g. the first and the second functional groups are interchangeable as to the attached functional group. There is no limit to the functional groups contained in a molecule and thus one or more conjugation reactions are possible between a pair of molecules and/or substances.

There are many methods for conjugation of two molecular entities, and the basic requirements for practical usefulness are: (a) the resultant conjugate is suitable for further applications, (b) conjugation reaction sites should be easy to prepare, (c) the reaction should cause minimal side and/or non-specific reactions, and (d) reaction time should be reasonably short. In the present invention four types of beads and the corresponding chemistry are preferred: gold (nanometer, thiol group), streptavidin coated magnetic beads (<10 μm, biotin group), TentaGel beads (Rapp Polymere GmbH, Germany, 10 μm, NH2 or OH conjugation chemistry), Sephadex beads (˜25 μm, used by 454 Sequencing technology, NH2 conjugation chemistry). Streptavidin coated magnetic beads are widely used for separation of different sequences through biotin-tag selection; the method is useful for purification, enrichment, separation, and other applications. Biotin functionalization of oligos may be accomplished by using standard phosphoramidite chemistry using a biotin-modifier agent. (Glen Research). This is a phosphoramidite agent and thus it can be coupled to the 5′-OH of an oligo after the full-length sequence is synthesized. Certain biotinylation agents permit coupling of a fluorescent dye after the biotinylation agent is coupled to the surface oligos. Such a fluorescent label can be used to validate the incorporation of the biotin moiety. Fluorescein molecules can be as a monitoring tool for synthesis and therefore can provide guidance for optimizing the biotinylation reaction.

The present invention includes a method of making addressable probe nanobeads mixture wherein each nanobead is attached to a single type probe molecule comprising: a) synthesizing an array of probe molecules on a surface wherein the molecule has a first terminus and a second terminus and wherein the first terminus is attached to a spacer that is attached to the surface and the second terminus can be coupled to a first functional group; b) conjugating a functional group to the second terminus; c) coupling tagged nanobeads that have been derivatized with a second functional group to functional group on the second terminus of the probe molecule; d) removing the uncoupled tagged nanobeads from the surface; e) capping the functional group of the uncoupled probe molecules; f) cleaving the tagged probe nanobeads from the array to form a mixture of addressable probe nanobeads mixture wherein each nanobead is attached to a single type probe molecule. The arrays of the present invention may comprises more than 1000 different probe molecules. In preferred embodiments the spacer has from 6-30 chemical bondsand is coupled to a cleavage site such that the addressable probe nanobead can be cleaved from the surface. Functional groups can be but are not limited to biotin, hydrazine, alkynyl, alkylazide, amino, hydroxyl, thiol, aldehyde, phosphoinothioester, maleimidyl, succinyl, succinimidyl, isocynate, ester, strepavidin, avidin, neuavidin and biotin binding proteins. Nanobeads can be treated with protein and surface blocking solution (such as 0.5% BSA in PBS buffer) to prevent non-specific binding before conjugation with the probe. Blocking proteins or non-ionic surfactants can be used to reducethe background non-specific interactions. A stringency wash step can be carried out using diluted reaction solution or a solution with increasing dissociation power. This further removes the beads retained on surface due to non-specific interactions and increases the ratio of correctly conjugated beads to non-specifically bound beads. The various reaction conditions, (e.g. buffer, solvent, temperature, pH and time) may have significant effects on the conjugation reaction. In preferred methods of the present invention the probe is preferably DNA oligonucleotides of 10-200 residues, and/or RNA oligos of 10-200 residues, and/or DNA and RNA chimer (mixes composition of DNA and RNA) 10-200 residues.

Functionalization can be accomplished by chemical conjugation. One widely used method is to generate an amino group such as by incorporation of an amino modifier or a 5-(3-aminoallyl)-dU into the oligo sequence or coupling an amino-linker moiety (FIG. 5) to the 5′-OH group using a phosphoramidite (Glen Research). The 5′-terminal amino group of the oligos can react with an activated ester, such as an NHS ester coated on the surface of beads to form an amide bond. The conjugate oligo-bead is stable in most chemical and bioassay conditions. The functionalization does not necessarily require the 5′-terminal amino group of oligos; else where in the oligo chain, suitable modifications as discussed for conjugation chemistry in the prescribed invention can be incorporated. Intermolecular conjugation linkage can be formed between the modification groups.

In an another embodiment of the present invention, functionalization can be accomplished by an adsorption method. The oligo can be modified, using 5′-thiol modifier (Glen Research), to a thiol group such that the oligo contains a SH moiety. SH has high affinity to gold surfaces. Gold spheres containing immobilized oligos have been successfully applied in assays of DNAs and in nano-structure constructions. Preferred functionalization chemistries are compatible with oligo synthesis/deprotection chemistry and these functional groups are commonly used as modifiers for oligo immobilization onto solid surfaces. The surface linkage chemistry suitable for synthesis and also removal of bead-tagged oligonucleotides from surfaces may be optimized to improve the efficiency of the generation of probe bead mixes.

The present invention also relates to methods for the conjugation reaction of a surface and beads which are in solution. In one embodiment of the present invention, the bead surface is derivatized with oligoethylene glycosyl amino spacer group. The total chain length of the spacer measured by number of bonds is greater than 6, and preferable is greater than 18 and more preferably greater than 30. The beads in coupling reaction solution (DIC/DMAP (1,3-diisopropylcarbodiimide/dimethylaminopyridine) in DMF/CH₂Cl₂) contain surface succinyl which can react with the surface linker. After the reaction, the beads are retained on the surface when the surface is washed multiple times. In comparison, the beads which do not have the surface succinyl group are washed away since there is no covalent bond formed between the beads and the surface.

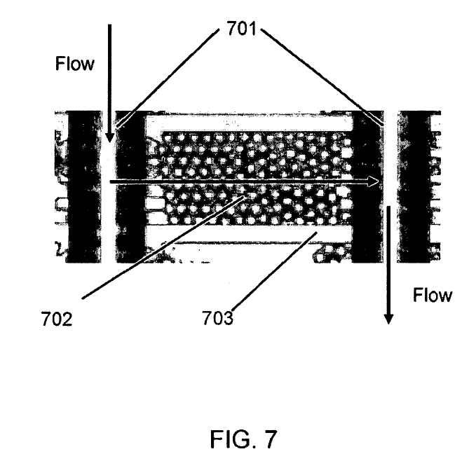

In an embodiment of the present invention, the surface to which the beads are attached is comprised of three dimensional reaction chambers as depicted in FIG. 1 and FIG. 7. The beads are adhered to the reaction chambers through conjugation reaction with the chamber surface so that they are not stripped from the surface as fluid flows through the channel (FIG. 7, 701) to chambers during multiple steps of chemical synthesis reactions (FIG. 7, 702). The beads are also confined to the chamber by the separation walls on both sides of the chamber aligned orthogonal to the flow channel (FIG. 7, 703). The methods of the present invention also provides for optimization of bead surface functionalization, thereby providing high quality synthesis results. The reaction chamber dimensions are 10 to 500 microns, which are larger than the bead sizes(10 nm to a few hundred μm) such that a large number of beads can be immobilized in each reaction chamber such that sufficiently large numbers of molecules (e.g. fmol to nmol, preferably pmol to nmol) are synthesized per array synthesis.

In one preferred embodiment of the present invention, FIG.1 depicts a three dimensional microfluidic pico-array device comprising three dimensional reaction chambers each having a surface area of approximately 90×180 mm² and a height of 16-30 μm. The array illustrated in FIG. 1, contains 3,968 reaction chambers that can accommodate 3,968 independent synthesis reactions. Based on the above referenced dimensions for the reaction chamber and the use of 1 μm beads filling 20% of the reaction chamber capacity each reaction site can accommodate about 8,100 or more beads.). At this level, one chip synthesis can generate beads for several hundreds to at least one thousand assays at pmol level.

It is realized that on a glass plate synthesis device (FIG. 2), probe synthesis is not restricted to a chamber for beads to be attached to the surface (FIG. 6) or probes cleaved to be used as a mixture of molecules or probe beads after attaching the cleaved molecules to beads added to the probe solution.

Depending on the size of the beads and the application an array having reaction chambers of this size can accommodate millions of beads. The microfluidic device can be scaled to increase or decrease the size of the reaction chambers according to application requirements. In a preferred embodiment the synthesis of molecules on the attached beads is performed using projection light which is digitally controlled and reaction reagent (PGR) forms under light irradiation (Gao X., et al., U.S. Pat. No. 6,426,184, Gao X., et al., U.S. Pat. No. 7,235,670; herein incorporated by reference). The light triggers chemical reaction on beads in the reaction chambers which are irradiated. Biopolymers may be synthesized by repeating the steps of light irradiation, deprotection, and coupling reactions. Beads conjugated to an array chip synthesis device is shown in FIG. 7 where 10 μm TentaGel beads were loaded on to a microfluidic chip in a dispersed mode, and the beads were reacted with a succinyl group on the chip surface thereby immobilizing the beads on the chip surface. The optical unit power for delivering suitable light strength and fluidic delivery for reactions occurring in reaction chambers filled with nanobeads need to be tailored to array synthesis. In general, irradiation power in the range of tens of mW to hundreds of mW at the position of the synthesis surface is desirable; sufficient amount of photogenerated reagents formed for the deprotection reaction.

In the present invention, one of the applications of the methods of making molecules on beads contained within an array is to increase the yield of the molecules. Present arrays can only make about 1 fmol of oligomer per reaction chamber. With the bead synthesis methods of the present invention about 1 pmol to about 20 pmols per reaction chamber can be produced. Furthermore with an array structure about 4,000 to about 100,000 different DNA oligos of these quantities can be made per array. The increased capacity allows researchers to utilize subsets of probe bead oligos to focus sequencing results on the areas of particular interest.

In the present invention, one of the applications of the methods of making molecules on beads contained within an array is to increase the yield of the molecules. In an embodiment of the present intention, one reaction site uses pseudo-codon (Gao, X. et al., WO2008/003100.) (pseudo-codon is a symbol, such as Z, which can represents more than one monomer building blocks in a synthesis, e.g. Z=A and G and this information is used for synthesis by a synthesizer. Adding a mixture of monomers to the synthesis results in formation of two or more compounds, depending on the number of monomers that the pseudo-codon includes. The use of multiple pseudo-codons results in formation of combinatorial libraries. For instance, for a oligomer synthesis, if the first pseudo-codon represents 3 monomers, and the second pseudo-codon represents 3 monomers, the synthesis of this oligomer results in a library of 9 different compounds). Thus, multiple different molecules can be made on a single reaction site. This form of synthesis is greatly benefit from the methods and devices of the present invention. The amount of each molecules in the library synthesis is greater than what obtained from a conventional synthesis.

In another embodiment, the present invention provides methods and devices for attaching beads to molecules that have been synthesized on a surface (FIG. 7). The molecules to which the beads may be attached include but are not limited to DNA, RNA, PNA, lipids, peptides, proteins, and carbohydrates. The bead may be attached by functionalizing a position or multiple positions on the terminus or within the molecule to generate a reactive site capable of affinity binding or covalent bonding with a separate molecule or a bead. In the present invention the preferred method is to functionalize the terminus such as the 5′ end of an oligo) however functionalization may be selected at any position(s) on the molecule to be synthesized. A benefit of 5′-functionalization for oligomers is that synthetic failure sequences are capped after the last step of coupling and thus are no longer available for functionalization. The quality of the collected 5′-functionalized sequences is thus improved.

After cleavage the bead probes can be collected and formulated into a mix. In the case where oligo molecules are to be cleaved from the synthesis surface the oligos may contain several functional sites (FIG. 5. Each oligo contains at least one cleavage site [designated X, FIG. 5], a 5′-functionalization site [designated ( ) FIG. 5] and a bead conjugation site [designated (O), FIG. 5]. But, the functional groups are not limited to the terminal positions and are synthesized at different positions in the probe molecule. The cleavage site for releasing surface molecules into solution is specifically designed so that desired molecules can be obtained for further applications. But it is also possible to use a general base or acid condition to cause the detachment of the probe molecules from surface. It is also possible to use an enzymatic condition to cause detachment of the probe molecules from surface. The probe bead cleavage site should be stable under synthesis conditions. The probe bead cleavage site should be able to be cleaved after the oligos are synthesized. Normally, the cleavage of oligonucleotides synthesized on a solid support, such as controlled porous glass (CPG), is accomplished by liquid ammonia hydrolysis of an ester bond. However, in array oligo synthesis, the synthesized oligos should remain on surface for assay applications, and thus it is not practical to use the same surface linkage chemistry as used in CPG oligo synthesis. U.S. Pat. No. 7,211,654, (Gao X., et al., herein incorporated by reference) describes a method for cleaving oliogos from synthesis surfaces; incorporated by reference. The cleaved oligos have 3′-OH groups and the OligoMix™ thus generated has been used in a variety of applications, such as primers, cloning inserts for mutagenesis and siRNA sequence libraries. The rU chemical modification can be used in either nuclease enzymatic reactions or base hydrolysis conditions for cleavage. These reactions are compatible with conjugation bonds and complexes such as biotin-streptavidin or covalent amide linkages. In a preferred embodiment of the present invention, the probe bead oligos contain an rU linkage. The rU monomer phosphoramidite can be incorporated in the oligo synthesis on surface. The cleavage reaction conditions can be optimized based on the specific type of the probe bead mixes.

In general, reactions are more efficient if the surface face oligos are more “solution-like”. Therefore, in preferred embodiments of the present invention linker and/or spacers are utilized to achieve more efficient reactions. In one embodiment of the present invention, the linker unit is a propylamine. The spacer unit is flexible due to the chain length, Hexaethylene glycol may be used as building blocks for the spacer. Optimization of spacer length is achieved by comparison of sequence sets containing different spacer lengths at different reaction sites on the same chip. The detection of fluorescence signal strength gives information on spacers which produce efficient synthesis (they have stronger fluorescence signals).

In a process of preparing a bead probe mix which includes oligo synthesis (FIG. 6, 901 and 902), oligo functionalization (FIG. 6, 903), oligo bead conjugation (FIG. 6, 904) and bead probe removal (FIG. 6, 905). The probe bead mix which may contain a large number of different sequences may be used for various applications including target-specific sequencing and target specific amplification. The oligos can be capture-probes (i.e. to hybridize and subsequently the duplexes are removed from the sample or primer-probes (i.e. as PCR or other amplification method primers) for amplification of a specific genomic region, and for amplification of genes such as cancer-related genes.

The probe beads of the present invention may also be made by array synthesis (parallel and in large number of different sequences) of molecules as depicted in FIG. 6 (901 and 902), which are then cleaved from the synthesis surface and subsequently mixed and attach to beads through conjugation.

Probe beads created can be utilized in bead, preferably nanobead, tagging, labeling and sorting, nanoarray assembling and other applications where beads are used individually or as a set of mixtures. Bead tracking and sorting methods of the present invention provide flexible and diverse applications of nanobeads. Addressable nanobead arrays may be created by using sorted nanobeads or by bead-tagging and tag-detection. Methods of nanobead tagging include oligonucleotide coding of each bead, sequencing decoding and multi-fluorescent tags or internally optically coded beads used in a combinatorial fashion (this now can be handled as subsets by flow cytometry). These methods of tagging the nanobeads permit easily assemblage of custom, addressable nanoarrays according to user's designs. These nanoarrays generated by the method of the present invention provide much greater diversity than microarrays presently available.

The nanobead arrays or a mixture of probe beads of the present invention may contain mixed molecular beads. For instance, profiling or detecting a broad line of cellular proteins will provide key information for many biomedical tests. This is presently not possible since there are no tools which are capable of simultaneously detection of different proteins. However, the nanoarrays or a mixture of probe beads of the present invention provide an array with different molecular probes thereby enabling a method for simultaneous detection of multiple different types of molecules in a sample, such as nucleic acids and proteins. For instance, comprehensive detection of proteins may be achieved by a nanoarray of molecular probes consisting of DNA and RNA for detection of nucleic acid binding proteins, peptides as substrates for their cognate proteins and enzymes (e.g. kinases and proteases).

The methods and compositions of the present invention provide high quality synthesis of oligonucleotides on chip and also provide methods of monitoring the synthesis procedures. The monitoring provides for control and continuous improvement in the quality of oligos. Several methods are effective in evaluate the quality of synthesis. Direct fluorescence residue coupling in oligos of different lengths These reactions can be performed under low fluorescence concentrations to avoid saturation of the dye molecules on surfaceHybridization using well-characterized control sequences to obtain perfect match (PM) and mismatch (MM) ratios. Cleavage and sequencing of long oligos made on surface. Finally, capillary electrophoresis analysis of the single sequence synthesized on an array.

While the preferred methods of making the nanobead arrays and probe beads mixes of the present invention use Photogenerated Reagent (PGR) chemistry and microfluidic array (μParaflo®) technology, methods and devices of the present invention are applicable to a variety of current DNA microarrays, including the microfluidic picoarray platform (4,000-30,000 features on a single array), other low to high density microarrays, (40,000>1 million features on a single array), Agilent arrays (40,000-200,000 features), Affymetrix/Nimblegen arrays (250,000>1 million features), Febit arrays of Nimblegen-type technology (8,000-40,000), or BioDiscovery's glass plate arrays (>40,000 features) synthesized using PGA chemistry. All of these current technologies can be adapted to suitable bead-conjugation (with modification chemistry development) to generate comprehensive probe bead mix products. Beads utilized in the methods and devices of the present invention include those of different sizes (submicron to 30 μm) and made from different materials, including but not limited to gold, polystyrene, sephadex, and grafted polyethylene glycol and polystyrene. The bead-loading, surface interactions, specific affinity binding or covalent bonding may be systematically optimized to maximize the conjugation of beads to oligos and minimize side reactions. The probe beads obtained from the methods discussed are in smaller quantities in the amount of about 0.1 fmol.

In preferred embodiments of the present invention the beads in the chip are present in the form of a monodispersion. To achieve a monodispersion several factors should be considered. Solvents (e.g. dipole, density, viscosity, temeperature, etc.), solvent pH, and bead handling (concentration, method of mixing, open or closed surface, etc.) have effects on the creation of a uniform bead distribution on surface.

In some embodiments of the present invention it is desirable to maximize the number of sequences made per unit area. While an increased sequence density is not necessarily a positive factor for hybridization microarrays, for probe bead oligos, it is useful for increasing the copies of the Oligos synthesized so that more sequences can be recovered from a given area. Dentrimer phosphoramidites such as trebler (Glen Research, Trebler Phosphoramidte) is selected as one of such examples, which couples with a surface OH group and, after deprotection, generate three OH groups, which can subsequently couple with three phosphoramidite molecules in next reaction step. Measurement of the oligo yield generated (determined by fluorescein coupling to the 5′-terminus of the sequence) as a function of the generations of trebler coupling gives 3×3, 9 times of the original OH numbers. The dentrimer method is limited by the steps the dentrimer can add before surface molecules saturate the surface or before surface becomes to be too crowded.

In an embodiment of the present invention, the probes and probe beads are used to generate oligo library in the form of droplet. A solution is made at a concentration of about nM (nanomolar) so that each droplet contains one types of probe or probe bead. Using the instrument from RainDance (http://www.raindancetechnologies.com/applications/next-generation-sequencing-technology.asp). the droplet of the sample and the droplet of the specific oligonucleotides are mixed and the probes selected for enrich specific genetic regions are PCR primers to allow sequence-specific sequencing and other genetic analysis.

### EXPERIMENTAL EXAMPLES

### Example 1

Monodispersion of Beads on Chip

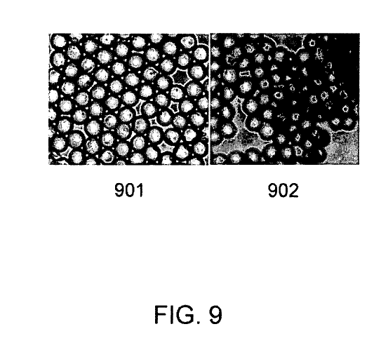

The experiment used 10 μm TentaGel beads (NH₂-derivatized) and different solvents (cyclohexane, acetonitrile, acetone, methylene chloride, tolulene, ether, ethanol, methanol, DMF, and DMSO). To each flat bottom vial, a trace amount of beads were dusted using a spatula. About 0.3 mL of solvent was added to the vial and the solvation of the beads were observed under a microscope and image was taken by a camera placed on the view port. FIG. 9, shows results of the beads in a mono-disperse mode (FIG. 9, 901, 10% tricholoroacetic acid in CH₂Cl₂) or in an aggregation state (FIG. 9, in ethanol, 902).

### Example 2

Bead Chip

A microfluidic chip fabricated to have 128×31 reaction cells connected by flow channels as shown in FIG. 1. The chip was put into a holder and at the inlet and out of the chip, the chip holder was connected with a 1/16 mm tube and luer lock. 10 μm TentaGel beads (NH₂ derivatized) in separate solvents: acetonitrile, methylene chloride, ethanol or its water mixture was slowly pushed into chip using either a syringe or a micro peristaltic pump at a rate of ˜50 μL/min. Image was taken from an epifluorescence microscope. FIG. 7 displays an image of an arbitrary reaction cell filled with the beads.

### Example 3

Surface and Conjugation Reaction

A glass surface was derivatized with oligos according to the method described in Gao, X. et al. 2001, (Nucleic Acids Re. 29, 4744-4750; herein incorporated by reference) and at the last step synthesis, biotin phosphoramidie was added and the coupling reaction in acetonitrile was 30 min. Following the reaction, glass surface was treated with 0.5% BSA (1 mL) in PBS, washed with PBS, and washing with CH₃CN, fluorescence streptavidin coated magnetic beads (Roche, 1 μm), was added to the bintinylated oligo surface and incubation was 1 hour. In a separate reaction, the glass plate without biotinylation derivatization was treated with the same procedures.

The plates with and without biotinylation were then thoroughly washed with acetonitrile and ethanol and images were taken using epifluorescence microscopy. Specific conjugation formation between biotinylated oligo on glass plate surface and fluorescence-tagged streptavidin was confirmed by fluorescence signal. The negative control using non-biotinylated glass plate and the streptavidin bead did not give fluorescence reading.

### Example 4

Biotinylated Oligos Conjugated with Strepavidin Beads on Surface and in Solution

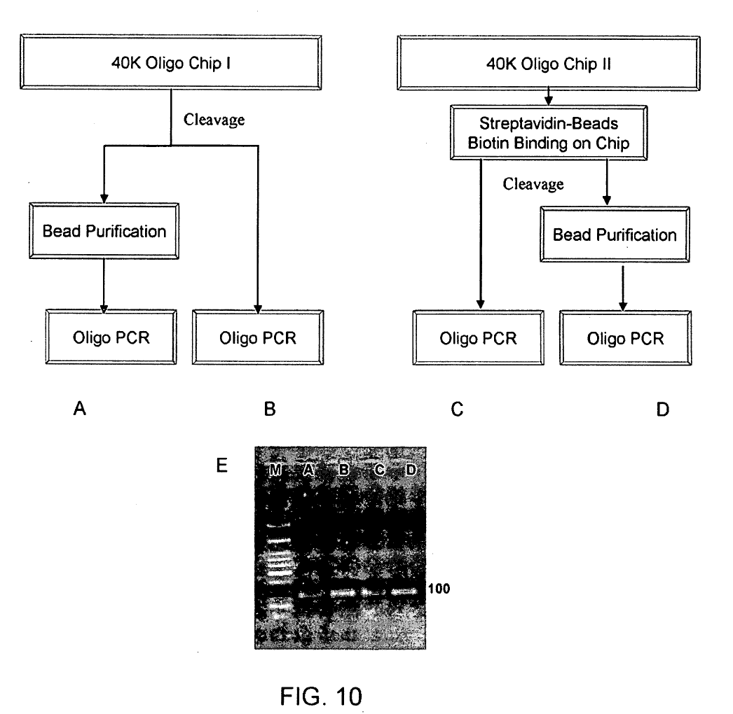

Two microfluidic chips containing oligos of average length of an average 40 nts plus primers (22 nts on either side) were used to synthesize oligos which have common primers for amplification (FIG. 10). The chips were synthesized using the method as described in Zhou, X. et al. 2004 (Nucleic Acids Res. 32, 5409-5417; herein incorporated by reference). After the last step of synthesis, biotin phosphoramidite was coupled to the oligo on chip. Chip I (FIG. 10) was treated with concentrated aqueous ammonia (300 μL, 55° C.) for 1.5 hour and the solution was collected and mixed with an additional 100 μL aqueous ammonia; this mixed solution was incubate at 55° C. for an additional 8 hours. The solution was evaporated and 0.2 mL binding buffer (10 mM Tris-HCl, 1 mM EDTA, 100 mM NaCl, pH 7.5) was added and the sample was equally spitted into two parts: A and B.

The streptavidin magnetic beads (0.1 mg/0.1 mL, (Streptavidin Plus Magnetic Particles, BD Biosciences)) was washed three times using Magnetight separation stand (MSS, Novagen) and binding buffer and the bead incubated with sample A for 30 min and washed with wash buffer (TEN1000: 10 mM Tris-HCl, 1 mM EDTA, 1 M NaCl, pH 7.5). The wash buffer collected as sample A.

Chip II (FIG. 10) was treated with streptavidin magnetic beads (0.1 mg/0.1 mL, washed three times with binding buffer before applied to Chip II). Oligos were cleaved from Chip II using RNase A (in 150 μl cleavage solution: 100 μg/mL RNase A; 500 μg/mL BSA; 2 mM EDTA, 20 mM K₂PO₄/KHPO₄ (pH6.2)) and cleaved oligos were divided into two samples C and D. Sample D was washed three times with washing buffer and the final collection of 100 μL is sample D.

Sample A, B, C, and D were used as template in the PCR reactions, PCR mix: 10 μL: 2 μL 10× PCR buffer, 5 μL of each primers (30-nts each, 10 μM), ˜2 μL template (samples A, B, C, D from the above process and originally from Chip I and Chip II), Vent polymerase (NEB), 74 μL biology grade water. PCR reaction began with heating sample to 95° C. for 2 min, the cycle consisted of 94° C. for 30 s, annealing at 56° C. for 1 min, extension at 72° C. for 30 s, 35 cycles. The reactions were stopped at 72° C. for 5 min.

The results of the four PCR reactions are shown in FIG. 10, E is an image of gel electrophoresis (2.5% QA-agrose high resolution gel, MidWest Scientific). The results, showing recovery of the biotinylated oligos through using streptavidin coated magnetic beads.

### Example 5

Immobilization Beads on Surface and Synthesis of Oligo

A glass surface derivatized with propylaminylsuccinylate (SU) was loaded with 10 μm TentaGel beads (NH₂-derivatized) in acetonitrile-pyridine, containing HOBt (60 mM) and DIC (2 eq.) at room temperature for 12 hours and then 40° C. for 71 hours. After thoroughly washed with acetonitrile, the plate was put into a DNA synthesis column (Expedite 8909), and placed in between two pieces of thin Teflon spacers. Oligo (5′ TAC ATA CCT CGC TCT) synthesis was carried out using a 1 μmol synthesis protocol on the synthesizer. The sequence was deprotected and cleaved off the glass plate surface using aqueous ammonia treatment at 55° C. overnight. The recovered oligo was analyzed and confirmed by HPLC analysis (260 nm peak) using reverse phase (C₁₈) column, equipped with photodiode array detector: gradient 1% TEAA (triethylaminonium acetate) in water and acetonitrile running from 5-5% in 2 min, 5-35% in 20 min, 35-100% in 5 min, 100-5% in 5 min, 5-5% in 2 min, at flow 1 mL/min.

1. A method of making probe bead mixture comprising:
a) synthesizing an array of probe molecules on a surface;
b) conjugating with functionalized beads to form probe beads;
c) cleaving the probe beads from the array to form a mixture of probe beads.

2. The method of claim 1 wherein the array probe molecules on a surface wherein the molecule has a functional group and the functional group can be coupled to functionalized beads;

3. The method of claim 1 wherein the functionalized beads are streptavidin coated beads

4. The method of claim 1 wherein the array probe molecules on a surface wherein the molecule has a biotin group and the biotin group can be coupled to strepavidin coated beads;

5. The method of claim 1 wherein the functionalized beads are gold sphere or gold coated sphere;

6. The method of claim 1 wherein the array probe molecules on a surface wherein the molecule has a thiol group and the thiol group can adsorb to gold sphere;

7. The method of claim 1 wherein the beads are nanobeads;

8. The method of claim 1 wherein the beads are microbeads;

9. The method of claim 1 wherein:
a) the uncoupled beads are removed from the surface;
b) the functional group of the uncoupled probe molecules are capped;
c) the cleaved probe bead mixture has each bead attached to a single type probe molecules.

10. The method of claim 1 wherein the array comprises more than 1000 different probe molecules.

11. The method of claim 1 wherein the probe molecule has a spacer from 2-120 chemical bonds.

12. The method of claim 1 wherein the probe molecule is coupled to a cleavage site such that the probe bead can be cleaved from the surface.

13. The method of claim 2 wherein the probe functional group is selected from the group consisting of biotin, hydrazine, alkynyl, alkylazide, amino, hydroxyl, thiol, aldehyde, phosphoinothioester, maleimidyl, succinyl, succinimidyl, isocynate, ester, strepavidin, avidin, neuavidin and biotin binding proteins.

14. The method of claim 1 wherein the bead functional group is selected from the group consisting of biotin, hydrazine, alkynyl, alkylazide, amino, hydroxyl, thiol, aldehyde, phosphoinothioester, maleimidyl, succinyl, succinimidyl, isocynate, ester, strepavidin, avidin, neuavidin and biotin binding proteins.

15. The method of claim 1 wherein the beads are treated with surface blocking solution to prevent non-specific binding before conjugation with the probe.

16. The method of claim 1 wherein the probe is DNA oligonucleotides of 10-200 residues;

17. The method of claim 1 wherein the probe is RNA oligonucleotides of 10-200 residues;

18. The method of claim 1 wherein the probe is DNA and RNA chimera or modified oligonucleotides of 10-200 residues;


SEQUENCE LISTING (8 sequences)

SEQ ID NO: 1 - DNA, 9 bp, Artificial Sequence
  Chemically Synthesized
cgtgcaatg

SEQ ID NO: 2 - DNA, 11 bp, Artificial Sequence
  Chemically Synthesized
tgcaatataga

SEQ ID NO: 3 - DNA, 10 bp, Artificial Sequence
  Chemically Synthesized
tacggattcc

SEQ ID NO: 4 - DNA, 11 bp, Artificial Sequence
  Chemically Synthesized
atgccaatgtg

SEQ ID NO: 5 - DNA, 12 bp, Artificial Sequence
  Chemically Synthesized
tctgtgcacatg

SEQ ID NO: 6 - DNA, 11 bp, Artificial Sequence
  Chemically Synthesized
tgcaatataga

SEQ ID NO: 7 - DNA, 11 bp, Artificial Sequence
  Chemically Synthesized
gtacgtgtacc

SEQ ID NO: 8 - DNA, 13 bp, Artificial Sequence
  Chemically Synthesized
acagctcactgtg